(19) United States
(12) Patent Application Publication
(10) Pub. No.: US 2017/0261427 A1
(43) Pub. Date: Sep. 14, 2017

(54) OPTICAL MEASUREMENTS OF CHEMICAL CONTENT

(71) Applicant: ANALOG DEVICES, INC., Norwood, MA (US)

(72) Inventor: Shrenik Deliwala, Andover, MA (US)

(73) Assignee: ANALOG DEVICES, INC., Norwood, MA (US)

(21) Appl. No.: 15/068,824

(22) Filed: Mar. 14, 2016

(51) Int. Cl. G01N 21/3577; G01N 33/18

(52) U.S. Cl. CPC G01N 21/3577; G01N 33/18; G01N 2201/062; G01N 2201/12

(57) ABSTRACT

Techniques for optical detection of target chemicals on/in samples are disclosed. Light of at least two different wavelengths, or different bands of wavelengths, interacts with a target chemical, and at least some of the light that has interacted with the target chemical is incident on at least two photodetectors. Each of the photodetectors is configured to detect light of a different wavelength, or a different band of wavelengths, that has interacted with the target chemical. A processing logic is configured to compute a ratio between a parameter indicative of the intensity of light detected by one photodetector and a parameter indicative of the intensity of light detected by the other photodetector, and to determine the presence and/or the amount of the target chemical based on the computed ratio. In this manner, a simple, compact, and non-contact optical measurement assembly for assessing chemical content using differential spectral measurements may be provided.

### TECHNICAL FIELD OF THE DISCLOSURE

The present invention relates to the field of optical measurements, in particular to systems and methods for evaluating content of water and other chemicals based on light absorption by the chemicals.

### BACKGROUND

Evaluation of water content is important in applications across a large variety of fields. For example, presence of water is often detrimental to the functionality or/and the efficiency of various devices such as e.g. fuel cells, photovoltaic devices, integrated circuit (IC) chips, etc. In another example, water content on the skin may be indicative of skin condition, such as e.g. skin dryness, and, therefore, be used in cosmetic or/and medical assessments. In yet another example, pharmaceutical industry also often needs to determine water content, e.g. in or on various chemical components. In still another example, soil moisture is also often of important and needs to be assessed.

Water has strong absorption peaks in many parts of the electromagnetic spectrum. Therefore, measurements of an absorption spectrum, i.e. variation in absorption vs. wavelength, may be used to determine the amount of water on or in a sample.

Some of the strongest peaks of water absorption are in the infra-red part of the electromagnetic spectrum. For that reason, many studies over the last 20 years have focused on spectroscopic measurements analyzing complete Near Infrared (NIR) spectra to measure the water content, especially focusing on strong absorption bands of water around 1450 and 1930 nanometer (nm) wavelengths. In these measurements, water content of a sample is correlated to the measured spectra using chemometric models which involve using principle component analysis, first or the second derivative spectra, etc. Spectrometer equipment used in these measurements is proven and works well. However, this equipment is also bulky and expensive.

Improvements in optical measurements of water content, as well as measurements of content of other chemicals, would be desirable.

### BRIEF DESCRIPTION OF THE DRAWINGS

To provide a more complete understanding of the present disclosure and features and advantages thereof, reference is made to the following description, taken in conjunction with the accompanying figures, wherein like reference numerals represent like parts, in which:

FIG. 1 illustrates a spectrum of water absorption;

FIG. 2 illustrates an example of a spectrum of a light source and spectra of transmission of two exemplary idealized optical filters, according to some embodiments of the disclosure;

FIG. 3 illustrates an apparatus for optical detection of a presence and/or an amount of a target chemical, according to some embodiments of the disclosure;

FIG. 4A illustrates relative positions of a sample and parts of an apparatus for optical detection of a presence and/or an amount of a target chemical in/on the sample for a reflection measurement, according to some embodiments of the disclosure;

FIG. 4B illustrates relative positions of a sample and parts of an apparatus for optical detection of a presence and/or an amount of a target chemical in/on the sample for a transmission measurement, according to some embodiments of the disclosure;

FIG. 5 illustrates an example of spectra of light reflected from samples containing different amounts of water, according to some embodiments of the disclosure;

FIG. 6A illustrates variation in the exemplary LED spectra of different LEDs;

FIG. 6B illustrates measured ratios for samples containing different amounts of water when different LEDs are used;

FIG. 6C illustrates ratios analogous to those shown in FIG. 6B but computed using normalized intensities, according to some embodiments of the disclosure;

FIG. 7 illustrates an example of a spectrum of a band-pass filter provided for the example of FIG. 5, according to some embodiments of the disclosure;

FIGS. 8A-8C illustrate how measurements can be made more robust to smooth changes in the background using weight parameters, according to some embodiments of the disclosure; and

FIG. 9 illustrates a flow diagram of an optical detection method, according to some embodiments of the disclosure.

### DESCRIPTION OF EXAMPLE EMBODIMENTS OF THE DISCLOSURE

Embodiments of the present disclosure provide optical measurement assemblies that are compact, substantially less complex, and relatively inexpensive compared to complex spectrometer equipment used in the prior art techniques described above. Optical measurement assemblies described herein may be used in any systems that require determination of presence and, possibly, the amount of a certain chemical component on or in a sample. Assemblies described herein may be especially attractive for, but are not limited to, cosmetic/medical, agricultural and industrial applications.

In some aspects, techniques for optical detection of a presence and/or an amount of a target chemical on/in a sample are disclosed. Light of at least two different wavelengths, or of two different bands of wavelengths, the light preferably originating from a single light source that is sufficiently broadband to cover both the wavelengths, interacts with a target chemical (e.g. is reflected from a sample comprising the target chemical, is transmitted through the sample, is partially absorbed by the sample, etc.), and at least some of the light that has interacted with the target chemical is incident on at least two photodetectors (i.e. on one or more photosensitive elements of each photodetector). Each of the photodetectors is configured to detect light of a different wavelength, or a different band of wavelengths, that has interacted with the target chemical. A processing logic is configured to compute a ratio between a parameter indicative of the intensity of light detected by one photodetector and a parameter indicative of the intensity of light detected by the other photodetector, and to determine the presence and/or the amount of the target chemical based on the computed ratio. In this manner, a simple, compact, and non-contact optical measurement assembly for assessing chemical content using differential spectral measurements may be provided.

Techniques proposed herein are described with reference to water being an exemplary target chemical of interest. However, these techniques are by no means limited to detecting presence and/or amount of water present, and can easily be extended to measurements of other target chemicals. Some examples of other commercially relevant analytes are briefly described at the end of the present disclosure.

Furthermore, techniques are described herein with reference to measuring content of a target chemical, where measuring content may include merely detecting presence or absence of the target chemical or may include assessment/evaluation of the amount of the target chemical present.

Still further, a description of a target chemical being present in or on a sample is to be understood that technique described herein may be applicable to measuring content of the target chemical only on the surface of a sample (i.e. “on a sample”), or within the outer-most layers of the sample, as well as to measuring content of the target chemical within the sample (i.e. “in a sample”). A person of ordinary skill in the art would immediately recognize considerations applicable to and modifications that may need to be done to the techniques described herein depending on whether the content is measured in or on a sample, all of which considerations and modifications being, therefore, within the scope of the present disclosure.

As will be appreciated by one skilled in the art, aspects of the present disclosure may be embodied in various manners—e.g. as a method, a system, a computer program product, or a computer-readable storage medium. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Functions described in this disclosure may be implemented as an algorithm executed by one or more processing units, e.g. one or more microprocessors, of one or more computers. In various embodiments, different steps and portions of the steps of each of the examples described herein may be performed by different processing units. Furthermore, aspects of the present disclosure may take the form of a computer program product embodied in one or more computer readable medium(s), preferably non-transitory, having computer readable program code embodied, e.g., stored, thereon. In various embodiments, such a computer program may, for example, be downloaded (updated) to the existing devices and systems (e.g. existing optical measurement modules and/or their controllers) or be stored upon manufacturing of these devices and systems.

Other features and advantages of the disclosure are apparent from the following description, and from the claims.

Basics of Optical Spectrometers

Spectrometers are devices that analyze intensities and other characteristics of received signals as a function of wavelength, frequency, energy, momentum, or mass in order to characterize matter (referred to in the following as “target chemical”). Optical spectrometers are spectrometers that analyze optical spectrum, i.e. distribution of frequencies or wavelengths, of electromagnetic radiation received at their optical input. Optical spectrometers are typically used to detect and quantify presence of various atoms and molecules in a certain region, substance, or material that the radiation passed through prior to being detected at the spectrometer. To that end, spectrometers measure intensity or/and polarization state of the received radiation as a function of a wavelength or any other variable indicative of the wavelength, such as e.g. frequency or energy of the received photons. Measurements may be carried out either in relative or in absolute units.

Spectrometer equipment currently used to characterize target chemicals is bulky, complex, and expensive. Therefore, improvements with respect to that are desirable, especially for low power, compact deployment for measuring particular chemicals.

Water Absorption

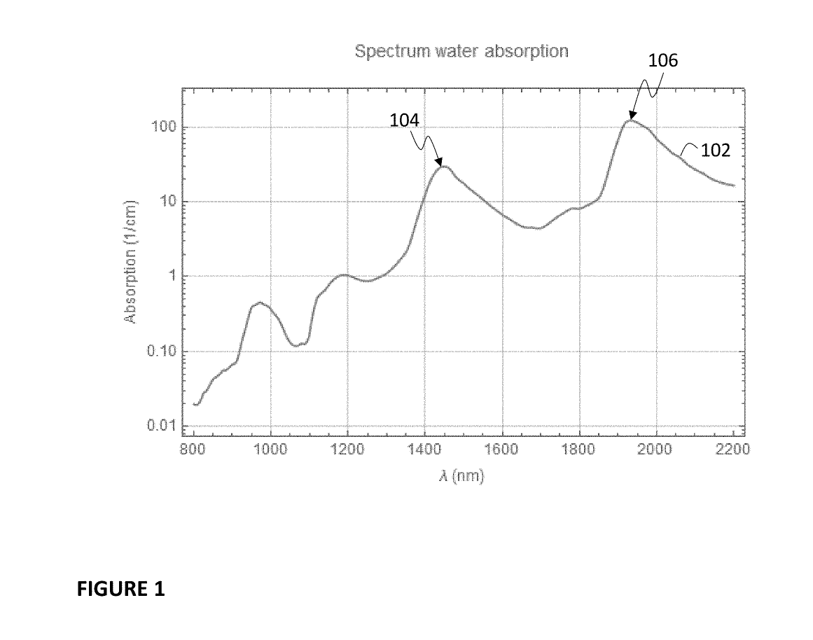

As previously described herein, water has strong absorption peaks in many parts of the electromagnetic spectrum. This can be seen in FIG. 1, where curve 102 illustrates water absorption at different wavelengths of light (i.e. a spectrum of water absorption). As can be seen in FIG. 1, the curve 102 has a relatively strong absorption peak at a wavelength of around 1450 nm, indicated as a peak 104, and another strong absorption peak at a wavelength of around 1930 nm, indicated as a peak 106.

Proposed Assembly for Optical Measurement of Target Chemical Content

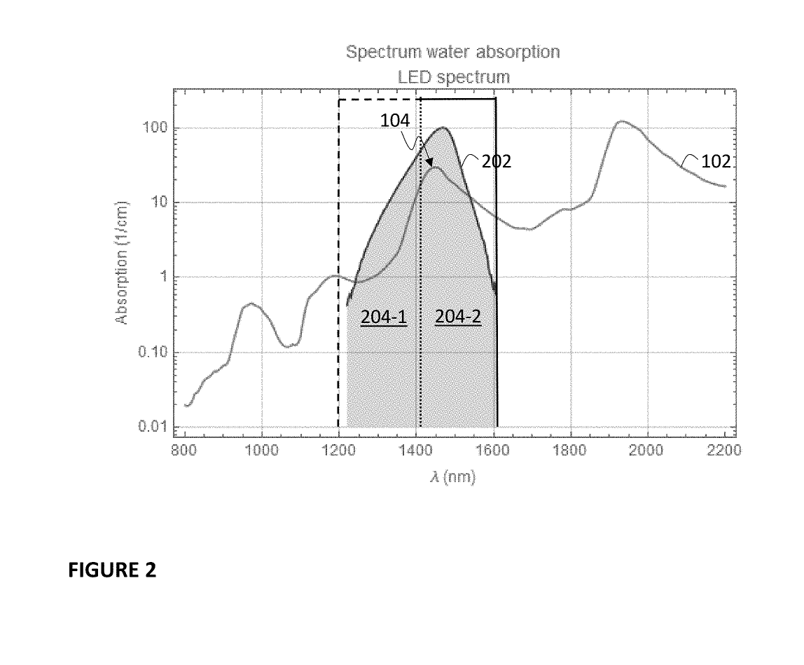

It has become relatively easy to find commercial light sources, e.g. light emitting diodes (LEDs), with various emission spectra. Therefore, commercial light sources with emission spectra that overlap absorption peaks of various target chemicals may be found. FIG. 2 provides the water absorption spectrum 102 as in FIG. 1, further illustrating a spectrum 202 of an exemplary light source, e.g. LED. As shown in FIG. 2, the emission spectrum 202 overlaps the absorption peak 104 of the water absorption spectrum. FIG. 2 also illustrates that a single light source can provide light at different wavelengths, or different bands of wavelengths.

Embodiments of the present disclosure are based on an insight that a ratio of measured light intensities of light of different wavelengths, or different bands of wavelengths, may be indicative of the presence and/or the amount of a particular target chemical. Two or more photodetectors can measure such light of different wavelengths or different bands of wavelengths, e.g. by being provided with a suitable filter that transmits light of only certain wavelengths to the photosensitive element of the photodetectors. FIG. 2 illustrates transmission wavelength bands 204-1 and 204-2 of two exemplary idealized filters that could be used to configure two photodetectors to detect light intensities in those bands.

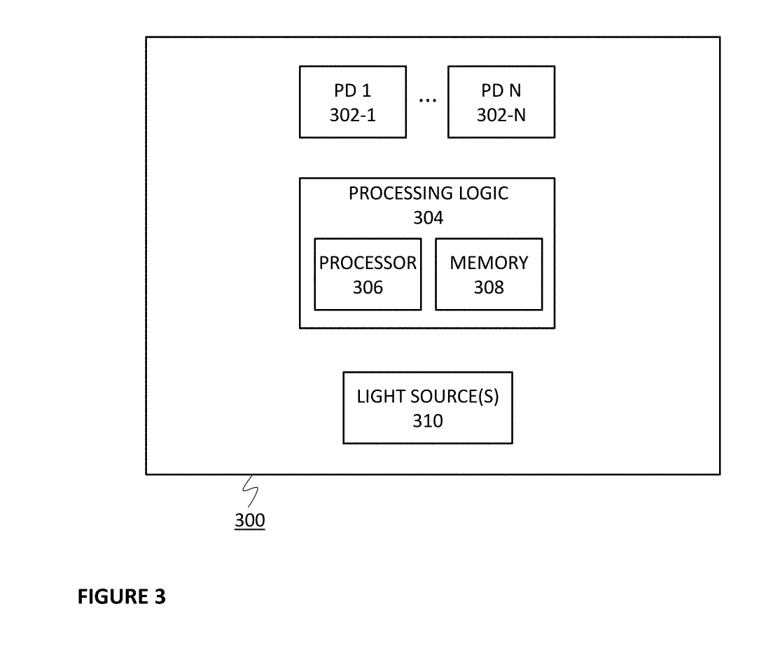

FIG. 3 illustrates an exemplary apparatus 300 for optical detection of a presence and/or an amount of a target chemical, according to some embodiments of the disclosure. The apparatus 300 may include two or more photodetectors 302, shown in FIG. 3 as a photodetector (PD) 1 302-1, and a PD N 302-N, where N could be any integer greater than 1. The photodetectors 302 are configured to detect light of different bands of wavelengths, where, in the context of the present disclosure, “different bands” imply that at least some of the wavelengths of one band are not included as the wavelengths of another band. Thus, two bands are considered to be different if e.g. they are partially overlapping, or if one band is included within another band (i.e. the wavelengths of one band are a subset of wavelengths of another band).

The photodetectors 302 may include any suitable photosensitive elements configured to generate an electrical signal, typically a current signal or change in resistance, in response to the light impinging onto the photoactive material of the photosensitive elements of the photodetectors. As with any detectors, the choice of a type of photodetectors used depends, first of all, on the wavelengths of radiation that each photodetector should be able to measure. For example, in the 0.2-1.1 um spectral range (i.e. a range of radiation having wavelengths between 0.2 and 1.1 um), silicon (Si) photodetectors could be used. However, due to the energy-band structure of silicon, Si photodetectors are not suitable for detecting radiation of wavelengths beyond 1.1 um and that's where e.g. water has the strong absorption peaks that could be exploited. Instead, germanium (Ge) photodetectors could be used for detecting radiation of wavelengths beyond 1.1 um and up until about 1.7 um. In fact, due to the energy-band structure of germanium, Ge photodetectors can be used for detecting radiation in the 0.7-1.7 um. For detecting radiation in spectral regions with wavelengths above 1.7 um, other types of detectors could be used such as e.g. InGaAs, InAs, PbS, InSb, HgCdTe, PbSe, GeAu, thermistors, bolometer, thermocouples or pyroelectric detectors.

Different photodetectors 302 could be configured to detect light of different bands by providing appropriate optical filters that filter light that reaches the photosensitive elements of the photodetectors. For example, for the example shown in FIG. 2, photodetectors each of the photodetectors PD 1 and PD2 could be e.g. a Ge photodetector provided with a respective band-pass optical filter so that PD1 then detects wavelengths in the band 204-1, while PD 2 detects wavelengths in the band 204-2. An optical filter may be provided in the form of a coating of a suitable material provided over the photosensitive region of the photodetector, as known in the art.

Each of the photodetectors 302 is configured to detect light that has interacted with the target chemical, e.g. by being reflected from, transmitted through, or partially absorbed by the sample comprising the target chemical. Optionally, as shown in FIG. 3, the apparatus 300 may also include one or more light sources 310 for generating light that interacts with the target chemical and is then detected by the photodetectors 302. Alternatively, the one or more light sources 310 may be provided externally to the apparatus 310.

In various embodiments, the light source(s) 310 may comprise a light emitting diode (LED), or any suitable component(s) for emitting light. The light emitted by the light source(s) 310 can be of any suitable wavelength (or a range of wavelengths), depending on the application, as long as it includes the wavelengths or a range of wavelengths that are to be detected by the photodetectors 302 and based on which the ratios described herein are to be computed.

In order to compute the ratios, the apparatus 300 may further include a processing logic 304. The photodetectors 302 are communicatively connected to the processing logic 304 in that the results of the measurements by the photodetectors 302 can be provided to the processing logic 304. The processing logic is configured to compute a ratio (R) between a first parameter indicative of at least an intensity of the light detected by e.g. a first photodetector PD1 (Int1) (possibly indicative of a combination of intensities of the light detected by each of the first and second photodetectors) and a second parameter indicative of at least an intensity of the light detected by a second photodetector PD 2 (Int1), and determine the presence and/or the amount of the target chemical based on the computed ratio. To that end, in some embodiments, the processing logic 304 may include at least a processor 306 and a memory 308, as shown in FIG. 3, configured to implement and/or control various techniques for measuring content, described herein.

While FIG. 3 illustrates the processing logic 304 to be included within the apparatus 300, in other embodiments, the processing logic 304 may be implemented external to the apparatus 300, in which case the processing logic 304 may be configured to exchange data with the apparatus 300, in particular exchange data with the photodetectors 302 and e.g. control the light source(s) 310, remotely, via any appropriate communication channel. In other words, instead of being implemented within the apparatus 300 as shown in FIG. 3, the processing logic 304 may be external to the apparatus 300 and be communicatively coupled to the apparatus 300.

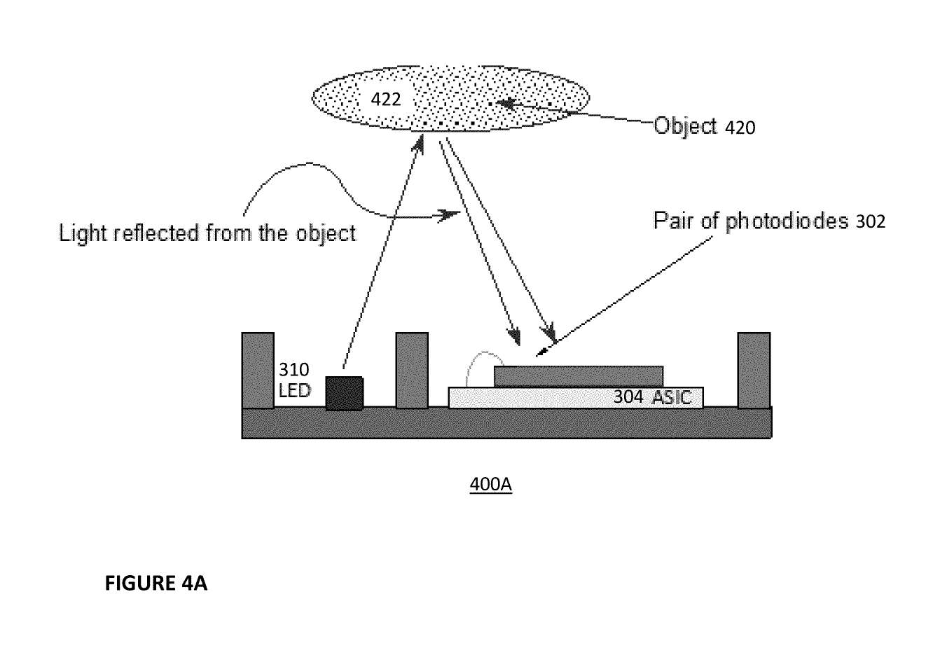

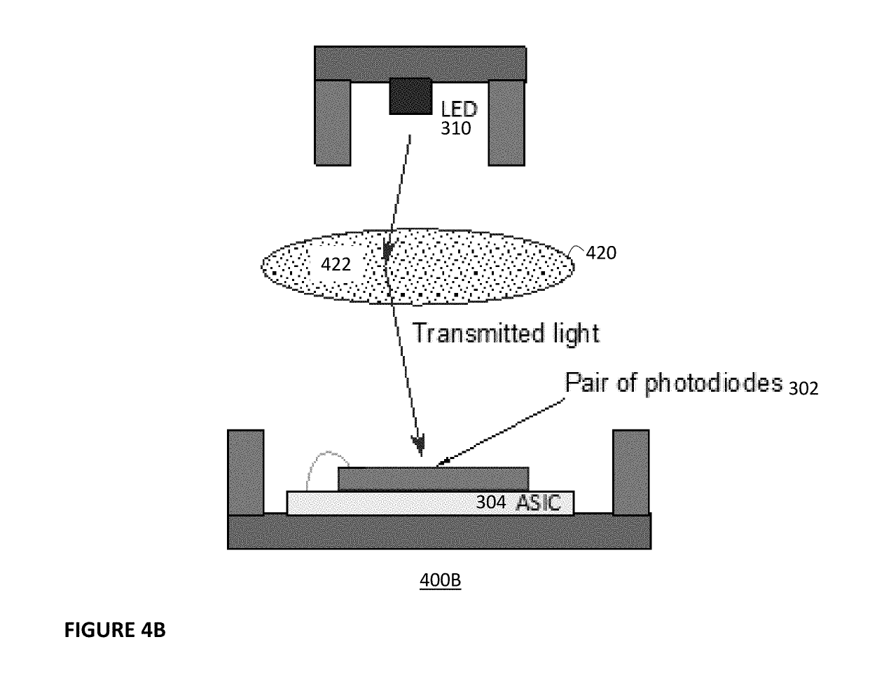

Two exemplary measurement systems such as the apparatus 300 are shown in FIGS. 4A and 4B. FIGS. 4A and 4B illustrate systems 400A and 400B where the photodetectors 302 are configured to measure light that is, respectively, reflected from and transmitted through an object 420 comprising a target chemical 422. Elements indicated in FIGS. 4A-4B by reference numerals shown in FIG. 3 are intended to represent the elements illustrated and described with reference to FIG. 3, which description is, therefore, not repeated here.

Examples of FIGS. 4A and 4B illustrate complete measurement assemblies including the light source(s) 310, photodetectors 302, and a basic processing logic 304 shown, in these examples as an Application Specific Integrated Circuit (ASIC). In the embodiments shown in FIGS. 4A and 4B, the photodetectors 302 are shown to be mounted on top of the processing logic 304. The mounting of detectors on top of the processing logic is not necessary for the principles disclosed here but is shown here to illustrate a compact system. In other embodiments, the photodetectors 302 may be provided in any other location with respect to the processing logic 304.

In some embodiments, another feature that would contribute to providing a compact system and could, optionally, be implemented is to place the photodetectors 302 in relatively close spatial proximity to one another, e.g. less than 5 mm apart. Not only would that enable a compact configuration but it would also help ensuring that the sampled reflected or transmitted light is substantially identical (i.e. that the fields of view of the photodetectors 302 overlap, at least partially), which would be advantageous because it could reduce or eliminate the issue of sampling spatially inhomogeneous scattered light from an irregular object.

In some embodiments, another configuration that would be compact and enable the photodetectors 302 to sample substantially the same light would be to interleave photosensing regions of the different photodetectors. Interleaving could ensure that the measurements by different photodetectors are spatially uniform.

Placing the photodetectors in close proximity to one another or/and interleaving their photosensing regions could eliminate the use of expensive fiber optic probes used in some prior art implementations for similar measurements. For example, in agricultural industry, it is often considered important to measure the water content of the fruits and vegetables to measure its freshness. It is often done by using a spectrometer. In order to measure the water content, it is also important to sample a sufficiently large patch on the surface of a sample in which the reflected diffuse light is collected and brought into the spectrometer. Since spectrometers themselves are large bulky devices, one has to employ fiber-optic bundles or an integrating sphere in order to carry light from the sample to the input optical port of the spectrometer. Since in some embodiments of the present disclosure, a compact system that uses an LED source and detectors that are in close proximity to one another, and the photodetectors directly receive the diffuse reflected light from the sample, at least these embodiments avoid the bulk and expense of the spectrometers and light transport.

In some embodiments, the photodetectors 302 could be provided on the same wafer and even on the same die of a wafer. The latter would be particularly advantageous because, with the advances of semiconductor Integrated Circuit (IC) fabrication technologies, there is an ever-increasing drive to integrate devices of various functionality on a die. In general, the term “die” refers to a small block of semiconductor material on which a particular functional circuit is fabricated. An IC chip, also referred to as simply a chip or a microchip, sometimes refers to a semiconductor wafer on which thousands or millions of such devices or dies are fabricated. Other times, an IC chip refers to a portion of a semiconductor wafer (e.g. after the wafer has been diced) containing one or more dies. In general, a system is referred to as “integrated” if it is manufactured on one or more dies of an IC chip. The system 300, or at least some portions thereof, could be provided as an integrated system.

As shown with FIGS. 4A and 4B, measurements could be performed either in reflection or in transmission. FIG. 4A illustrates that light generated by the light source 310 is reflected off of the object 420, thereby interacting with the target chemical 422 in/on the object (target chemical shown as dots in the object 420), and the reflected light is incident on the photodetectors 302. FIG. 4B illustrates that light generated by the light source 310 is transmitted through the object 420, thereby interacting with the target chemical 422 in/on the object, and the transmitted light is incident on the photodetectors 302.

In various embodiments, ratios between parameters indicative of intensities measured by the different photodetectors 302 could be defined in different manners. Some of considerations for choosing a suitable ratio definition include e.g. a particular configuration of the system, which primarily depends on the optical filters applied to the photodetectors or bands of the wavelengths that the photodetectors are configured to measure, a particular target chemical of interest, and the sample material in/on which the target chemical of interest is provided. Inventor of the present disclosure realized that by directly comparing the output of the different optical detectors relative to one other and forming a calculated output, with a particular ratio between parameters indicative of intensities at the output of the photodetectors being one of the simplest possibilities, one can make the calculated output to be independent of the light source intensity, variations background material, gain of the system, as well as changes introduced in the light source as well as receiver electronics due to e.g. temperature. Similar performance is achieved using complex and bulky full spectroscopy systems by creating a model based on 2^{nd} derivative of the spectra etc.

Some exemplary ratios are now described. Based on these examples, a person of ordinary skill in the art would be able to envision implementations using other ratios and other optical filters for the photodetectors 302 to measure content of target chemicals, all of which other implementations being within the scope of the present disclosure.

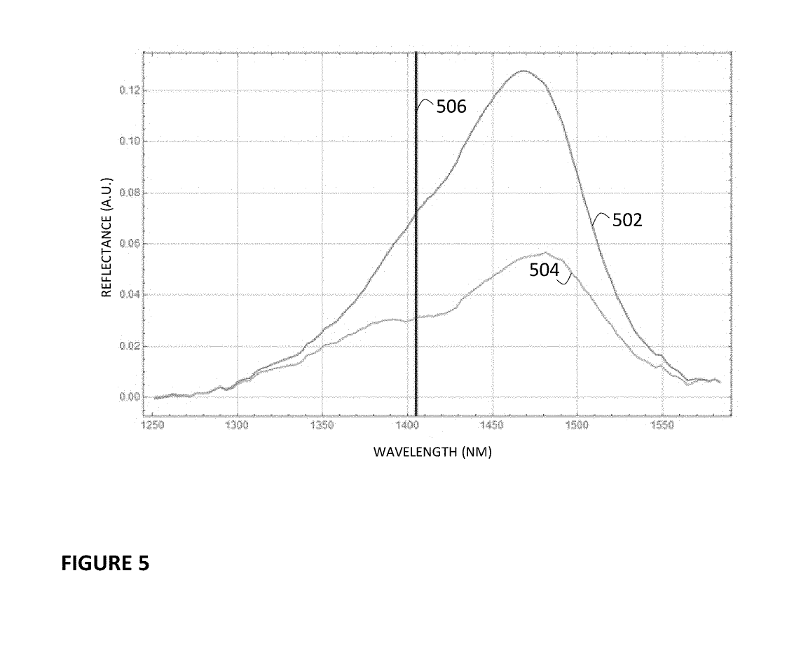

In one example, a spectrum of light generated by the light source(s) 310 and reflected from the object comprising a target chemical may be divided into two parts by applying appropriate optical filters to photodetectors PD1 and PD2. For example, the optical filters could be such that PD1 measures a part of the spectrum shown as 204-1 and PD2 measures a part of the spectrum shown as 204-2 (i.e. band-pass filters are applied). In other examples, the optical filters could be complementary to one another in that one photodetector measures all light that it can measure below a certain wavelength and another photodetector measures all light that it can measure above a certain wavelength, as e.g. shown in an example of FIG. 5 where a line 506 indicates a wavelength (1425 nm in the example shown) that differentiates the two filters. In such examples, by choosing an appropriate filter characteristics to divide the light source spectrum into the two parts, one can directly measure the absorption by a target chemical, e.g. water, by taking a ratio of the measured light intensities. In various embodiments, a ratio may be computed as e.g. .g.

$$R = \frac{Int 1 - Int 2}{Int 1 + Int 2} , R = \frac{Int 1 + Int 2}{Int 1 - Int 2} , R = \frac{Int 2 - Int 1}{Int 2 + Int 1} , R = \frac{Int 2}{Int 1} , \\ R = \frac{Int 2 + Int 1}{Int 2 - Int 1} , or R = \frac{Int 1}{Int 2} ,$$

where Int1 is a variable representative of an intensity measured by a first photodetector PD1 302-1, and Int2 is a variable representative of an intensity measured by a second photodetector PD2 302-2.

Such ratios would, advantageously, be independent of the absolute light intensity detected by the photodetectors and, hence, independent of the gain or the efficiency of the system including electronic gain, distance to the sample, sample orientation, etc., which would allow for a more accurate measurement. Presence and amount of water may then be determined based on the computed ratio. A simple experiment using a full spectrometer will now be described, the experiment illustrating that it is indeed possible to distinguish water, or other target chemical, content using ratios are described above. Theoretical and practical frameworks as to how to use ratios described herein to measure content are described thereafter.

FIG. 5 illustrates dry and wet spectra of light reflected from a sponge, as measured with a spectrometer and an LED centered around 1450 nm. In FIG. 5, line 502 illustrates spectrum for light reflected from a dry sponge and line 504 illustrates spectrum for light reflected from a wet sponge. In this case, a full spectrometer was used to illustrate the changes in the spectrum caused by the presence of water.

Line 506 in FIG. 5 indicates that, if the spectra were measured by two photodetectors where one photodetector (e.g. PD1) measured the total light for all wavelengths less than 1405 nm (Int1) and another photodetector (e.g. PD2) measured the total light for all wavelengths greater than 1405 nm (Int2), then the ratio of the output of these two detectors can be directly related to the water content of the sponge. For example, for the case shown in FIG. 5, a simple ratio

$$R = \frac{Int 2}{Int 1} ,$$

as described earlier, would be 3.56 in case of a dry sponge and 2.56 in case of a wet sponge with a certain amount of water, illustrating that the ratio directly provides the ability to discriminate between wet and dry sponges. The ratio will vary smoothly for sponges containing various amounts of water and, thus, could be used to determine, at least approximately, the amount of water present.

Change in the ratio from 3.56 to 2.56 in the example described above can be explained as follows. Of the two photodetectors used, it is the photodetector PD2 that measures light at the wavelengths where water has a strong absorption peak (i.e. the absorption peak at around 1450 nm). Consider that, for a dry sponge (i.e. no, or very limited amount of water present) the ratio

$$R = \frac{Int 2}{Int 1}$$

has a certain value. When the sponge is wet (i.e. more water present compared to the dry sponge), intensities measured by both PD1 and PD2 will be less than those measured by these photodetectors when the sponge was dry. In addition, because absorption is stronger in the range of the PD2, due to the strong absorption peak of water in the range of PD2, intensity measured by PD2 will decrease by a factor that is greater than that of intensity measured by PD1. In other words, compared to a dry sponge, Int2 will decrease more than Int1. Therefore, the ratio for a wet sponge is less than the ratio for a dry sponge, and is indicative of the presence and the amount of water present in the sponge. Similar reasoning applies to other target chemicals and to other ratios that may be computed when at least two photodetectors that measure different wavelength bands are used, because different wavelength bands result in different effects of absorption on the intensities measured by the photodetectors, which difference may then be used to assess presence of a target chemical.

Note in the above case, that the “dry” sponge mostly reflected the spectrum of the LED itself and hence the ratio was really the ratio of the LED spectrum as seen by the two detectors. The wet sponge simply modified this ratio as presence of water drastically changed the reflectance at different wavelengths within the spectrum of LED.

One way to relate computed ratios to the presence and/or the amounts of different target chemicals is to use theoretical models to predict what a particular ratio should be for a particular setting (i.e. given a certain light source spectrum, certain optical filters on the two or more photodetectors, a certain target chemical, and a certain sample/object in/on which the target chemical is provided). Basic physical description of the light propagation and radiation transfer theories support the fact that the absorbance of a sample is directly proportional to the amount of target chemical, and further that the diffuse reflectance of the sample is related to the absorbance. The radiative transfer models for both reflection and transmission are well known in the literature. For example, in transmission, the Beer-Lambert law is applicable, in which T=Exp(−A(λ)), where T is the transmission and A is the total effective absorbance (including the effect of scattering) at a particular wavelength. In reflection, more complex relationships are applicable, such as the theory of Kubelka and Monk in which the absorption coefficient is proportional to the measured reflectance

$$A ∝ \frac{{( 1 - R )}^{2}}{2 R} .$$

This illustrates that there exist relationships between measured transmitted or reflected light and absorbance and, therefore, relationships between measured transmitted or reflected light and the amount of target chemical in/on a particular sample.

Theoretical models can also take into consideration further variables that affect intensities of measured transmitted or reflected light, such as e.g. scattering. Scattering coefficients are often known for the materials of interest (not only the target chemicals themselves but also the sample/objects in/on which they are provided). Scattering is wavelength dependent in that different wavelengths have different scattering coefficients. For most materials, scattering coefficients change fairly slowly over the relatively narrow wavelength band from a light source such as an LED. For example, for the human skin, scattering coefficients have been measured and change as λ^{-0.22}, where λ is the wavelength of radiation being scattered. Thus, there will be interplay between scattering by the material of the sample or object and absorption by the target chemical, e.g. water, contained in the material. Radiative transfer equation that includes light scattering and absorption can relate the observed reflectance or transmittance to the absorbance and scattering coefficient of the material in the sample. In some embodiments, simplified models that relate observed reflectance or transmittance to the sample absorbance could be used, such as e.g. models based on modified Beer-Lambert's law or the Kubelka-Munk equation or many similar descriptions. In this manner, observed reflectance or transmittance can be mapped to the absorbance and further parameters such as e.g. scattering of a particular sample with a particular target chemical.

In practice, the actual spectra of the reflected or transmitted light will be fairly complex as they depend not only on the absorption by the target chemical but also on other variables, such as e.g. scattering particulate size in the material test and whether any direct light, e.g. specular reflection or directly transmitted, reaches the photodetectors. In some settings, it may be impossible or at least impractical to only use theoretically-derived relationships between the computed ratios and the amount of target chemical. Therefore, some embodiments of the present disclosure include performing a calibration of the measurement system 300 in order to relate computed ratios to the presence and/or the amounts of different target chemicals. As used herein, calibration refers to empirically determining, in a controlled, known, environment, one or more ratios for one or more target chemicals, provided in different known amounts, in/on particular samples/objects, so that these empirical measurement can later be used to measure unknown content for the target chemicals.

In some embodiments, one or more ratios as described herein could be measured, e.g. during the manufacture of the apparatus described herein, for certain known standards, e.g. certain samples/objects with known content of certain target chemicals. Results of the calibration would be stored in, or made accessible to the measurement system 300 in any other way, providing relations between target chemicals, ratios, amounts of the target chemicals present, etc. Subsequently, in operation (i.e. in the real, field, measurements), ratios computed based on the measured intensities as detected by the two or more photodetectors 302 are compared, e.g. by the processing logic 304, with the calibration results to assess the presence and/or amount of a certain target chemical present. For example, the quantity of the target chemical present can be determined from the computed ratio by plotting the ratios measured for different amounts of target chemical in a controlled environment (e.g. obtained during calibration) and then using the relationship thus developed, processing logic 304 can determine the presence and/or quantity of the target chemical based on the ratio value in the plot. Of course, in other embodiments, methods other than plotting may be used for relating ratio values to quantities of the target chemicals, all of which methods being within the scope of the present disclosure.

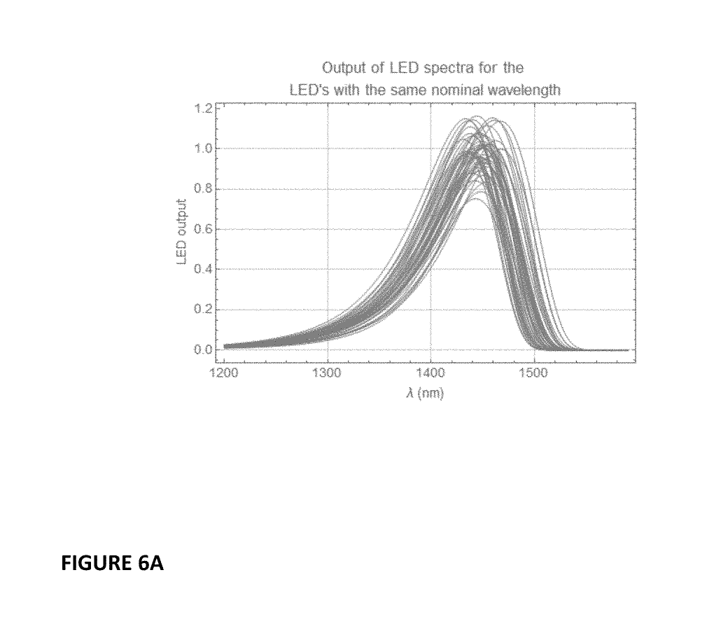

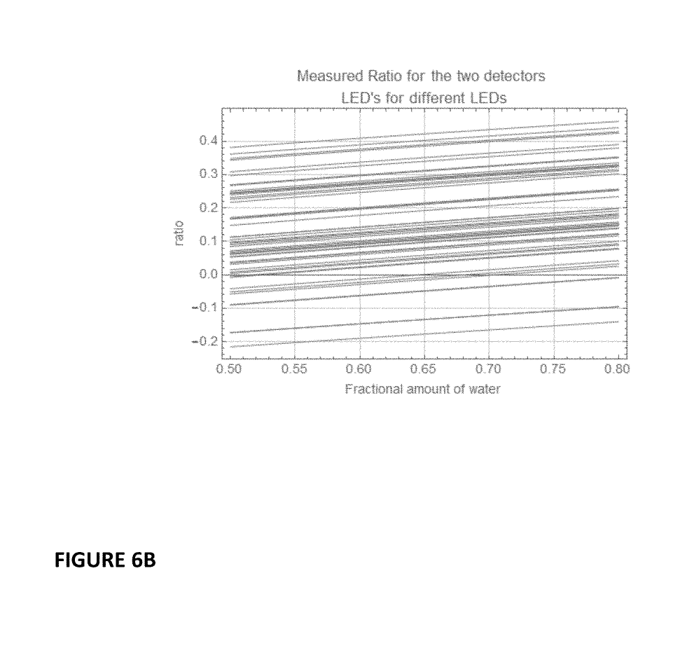

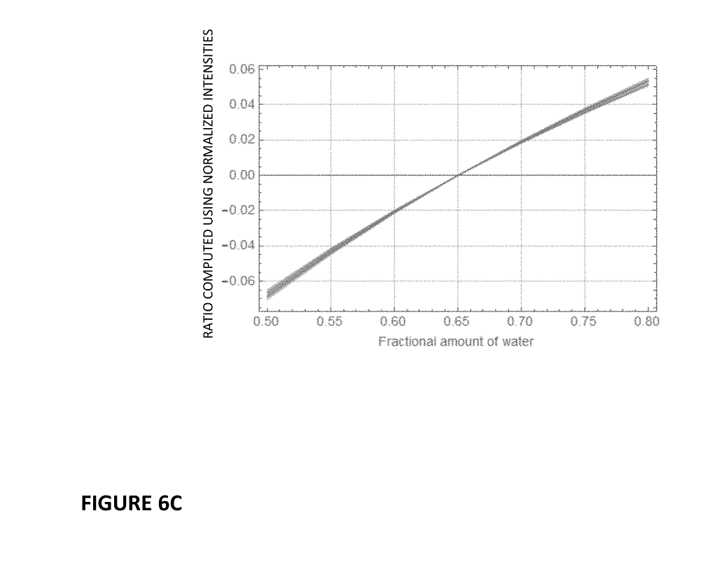

It should be noted that, in general, the shape of a spectrum of a given light source 310, e.g. an LED, will impact the measured ratio for a particular set of photodetectors 302. For example, FIG. 6A illustrates sample spectra of commercially available 1450 nm LEDs when they are randomly chosen from a manufacturing line. As can be seen in FIG. 6A, there is a substantial variation between different LEDs. A basic radiative transfer theory, such as e.g. the Kubelka-Munk relationship described earlier, may be used to estimate the impact of the variations in the LED spectrum on the ratios computed for a particular sample. This is illustrated in FIG. 6B, showing measured ratios for a system employing two detectors PD1 and PD2 as described above, for different amounts of water (different lines represent measurements for different LEDs of those LEDs shown in FIG. 6A). FIG. 6B illustrates that there are substantial variations in the measured ratios, depending on which particular 1450 nm LED is used. This means that, when using such light sources, while each particular measurement system may show a good relationship, the meaning of the relationship may be compromised across the different systems using LEDs with different emission spectra.

The above-described impact to the measurement can be mitigated by normalizing the intensities measured by the different photodetectors 302 during the field measurements to the intensities measured by these photodetectors during the calibration of that particular system 300. Thus, instead of using Int1 in computing a ratio, Int1/Int1_{cal} would be used, and instead of using Int2, Int2/Int2_{cal} would be used. Using such normalized intensities in computing a ratio makes the relationship of the computed ratio with the amount of a target chemical robust to variation in LED spectra. This is illustrated in FIG. 6C, showing ratios computed using normalized intensities for a system employing two detectors PD1 and PD2 as described above, for different amounts of water. Similar to FIG. 6B, different lines in FIG. 6C represent measurements for different LEDs of those LEDs shown in FIG. 6A. In contrast to FIG. 6B, FIG. 6C illustrates that variations in computed ratios for different LEDs are significantly reduced when normalized intensities are used for computations.

FIG. 6C illustrates ratios computed as

$$R = \frac{\frac{Int 1}{Int {1}_{cal}} - \frac{Int 2}{Int {2}_{cal}}}{\frac{Int 1}{Int {1}_{cal}} + \frac{Int 2}{Int {2}_{cal}}}$$

In other embodiments, other ratios described herein could be re-written using normalized intensities as described above. Conversely, all of the description provided herein with respect to intensities Int1, Int2, etc., represented as absolute intensities measured by different photodetectors are applicable to normalized intensities Int1/Int1_{cal}, Int2/Int2_{cal}, etc.

Calibration is particular advantageous for the embodiments that use normalized intensities in computing the ratio because intensities are normalized based on calibration measurements in controlled environment. Such an approach allows obtaining a high quality relationship between the content of a target chemical and the measurement even when the spectrum of a light source may vary substantially from one measurement system to another.

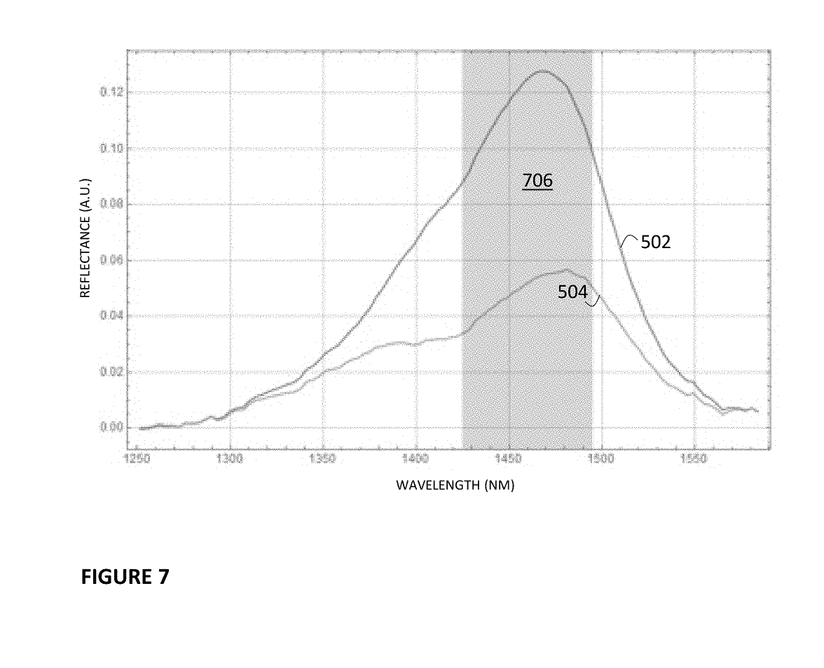

Using normalized intensities makes the measurements robust to spectral shape variations. In some embodiments, more advanced ratios may be computed or/and optical filters may be provided onto the different photodetectors such that smooth variations in the reflectance of the sample vs. wavelength, e.g. scattering induced reflectivity changes, are also suppressed in the measurements, making the measurements even more robust. In various embodiments of the present disclosure, optical filters on the photodetectors 302 can involve band-pass or band-stop filters, or/and at least one photodetector 302 could be provided without any additional filter. For example, a first photodetector PD1 could be provided with a band-pass filter and, thus, configured to detect light in a certain band, such as e.g. a band 706 indicated as a shaded area in FIG. 7, while a second photodetector PD2 could be left without a filter and, thus, configured to detect light in a band defined by the bandgap structure of the photosensitive material of the photodetector. This may be implemented by e.g. coating PD1 with a filter to simply pass light of specific wavelengths as shown with the range 706, while PD2 is left uncoated and thus collects light at all wavelengths that it can detect. In such an example, a ratio may be computed as

$$R = \frac{α Int 1 - β ( Int 2 - Int 1 )}{Int 2} ,$$

where α and β are predefined weight parameters which could be e.g. determined empirically or calculated based on one or more theoretical models.

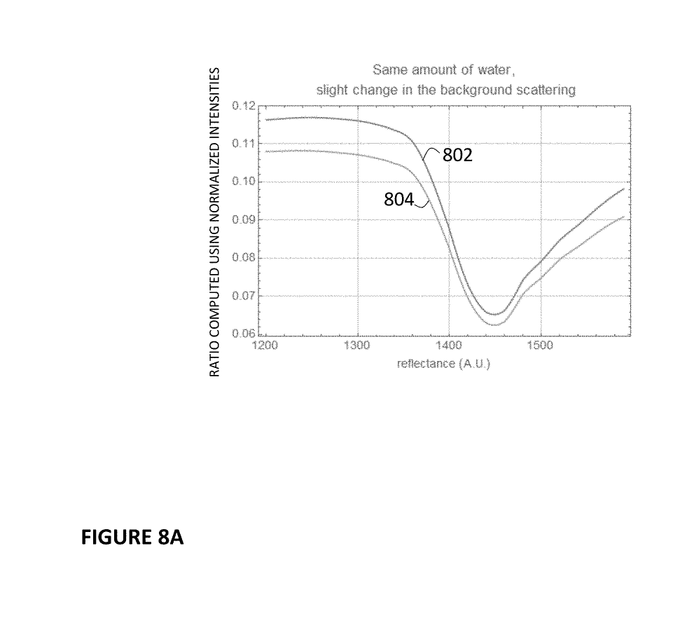

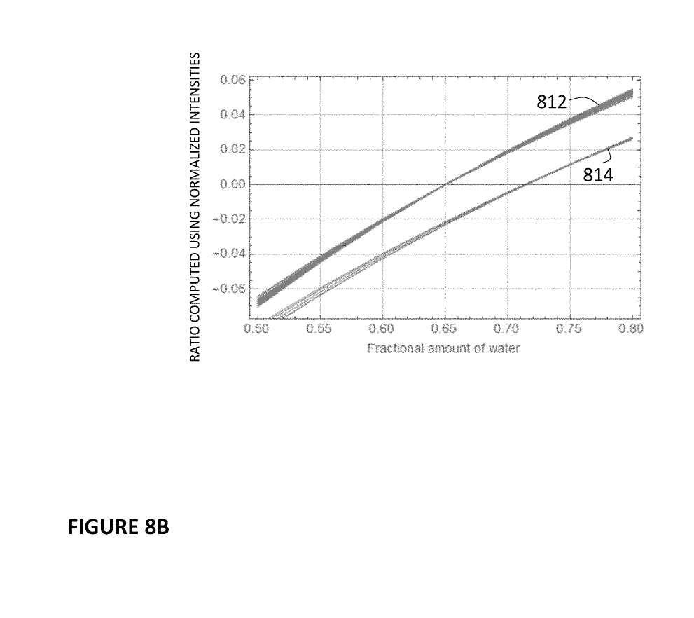

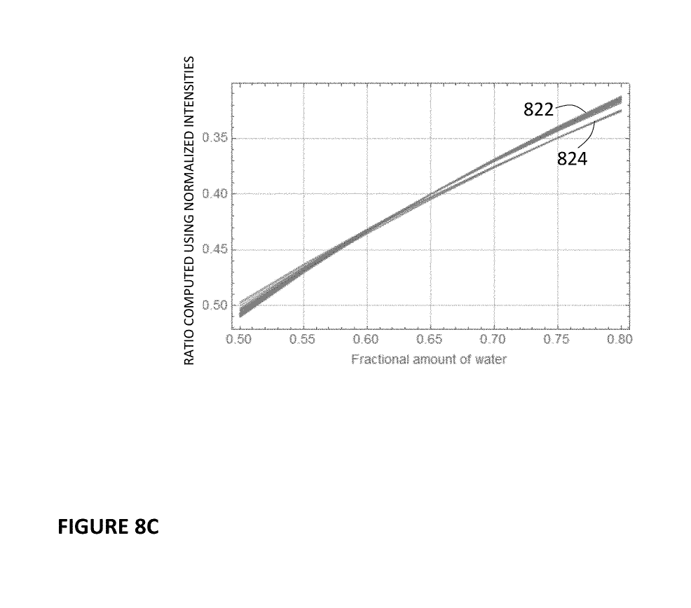

FIGS. 8A-8C illustrate how measurements can be made more robust to smooth changes in the background using weight parameters α and β as described above. For each of FIGS. 8A-8C, ratios were computed using normalized intensities (as shown with the y-axes), but in other embodiments, weight parameters can be implemented without using normalized intensities (i.e. using absolute values of the intensities). Two curves shown in FIG. 8A illustrate that, for the same amount of target chemical (water, in the example shown), there could be slight change in the background scattering. FIG. 8B illustrates that ratios calculated without using weight parameters α and β for slightly different background scattering properties of FIG. 8A. In particular, FIG. 8B illustrates a set of ratios 812 and a set of ratios 814. Both sets of ratios are computed using normalized intensities for a system employing two detectors PD1 and PD2 as described above, for different amounts of water, similar to the set of ratios shown in FIG. 6C. The set of ratios 812 corresponds to the scattering properties shown with the curve 802 in FIG. 8A, while the set of ratios 814 corresponds to the scattering properties shown with the curve 804 in FIG. 8A. FIG. 8C illustrates sets of ratios 822 and 824, similar to sets of ratios 812 and 824, respectively, shown in FIG. 8B, except that the ratios in the illustration of FIG. 8C are computed using appropriate weight parameters α and β (in the example shown, the weight parameters are equal to 1 and 1.4, respectively). FIG. 8C illustrates that variations in computed ratios for different background scattering parameters are significantly reduced when weight parameters are used.

Calculating ratios as described above may be viewed as a crude second derivative of the spectrum. As such, second derivative of a spectrum (vs. wavelength) is independent of gain, which was achieved by the first derivative and the ratios discussed in the first example, as well as any smooth variation in the absorption or reflectance across the wavelengths. Thus, the ratio becomes even more sensitive to the absorption spectrum of the target chemical.

Descriptions provided above could be generalized to embodiments where three measurements are taken, using three photodetectors configured to detect light in different bands. For example, three photodetectors 302 could be used for wavelength ranges that could be referred to, relatively, as high, middle, and low wavelength regions. Thus, in various embodiments of the present disclosure more than two photodetectors could be used and one or more ratios between parameters indicative of intensities measured by these photodetectors could be used. Such ratios could involve coefficients, similar to the coefficients α and β described above, which could be chosen, e.g. determined empirically or calculated based on one or more theoretical models, to provide the best sensitivity to the target analyte.

In some embodiments, the light source(s) 310 may be modulated and the photodetectors 302 may be configured to lock onto the modulation to reduce or eliminate contamination from the ambient light at the wavelengths detected by the photodetectors. Any modulation as known in the art could be used for this purpose, such as e.g. amplitude modulation, phase modulation, polarization modulation. The other very important advantage is that the LED may be switched very rapidly and the detected signal “locked” to the LED switching to eliminate the effects of the ambient light that may also fall on the detector.

In some embodiments, two or more of the photodetectors 302 could be configured to perform their measurements substantially simultaneously, or at least during overlapping times. This could be useful for eliminating any motion induced artifacts such as e.g. when measuring skin hydration and hands being unsteady or when measuring moisture in industrial process control and objects being measured moving on e.g. a conveyor belt.

Descriptions provided herein for water can readily be extended to any other chemicals of interest that have relatively narrow absorption bands and can, therefore, be assessed using techniques of the present disclosure. For example, fats or sugars may be measured in this manner. In various embodiments, the choice of bands that different photodetectors should be configured to measure, and of the light sources to use for illuminating the target materials would depend on the particular target chemicals and target materials expected, e.g. using some of the considerations described herein.

For example, for measuring water content, an LED centered at about 1460 nm or/and an LED centered at about 1930 nm could be used as the light source(s) 310. In another example, for measuring fat content, an LED centered at about 1200 nm could be used. In yet another example, for measuring protein content, an LED centered at about 1300 nm could be used. Using an LED centered at about 1726 nm would allow e.g. measuring sebum, which could be relevant for cosmetic industry and skin health applications. In other embodiments, a broadband light source may be used, e.g. a white light source, may be used. In some embodiments, an extended white light LED with phosphors that emit in the near infra-red region may be used as a light source, e.g. to measure oxygen level in the blood, a process referred to as pulse oximetry.

In still further embodiments, multiple systems such as the system 300 may be included or the system 300 may be provided with multiple sets of photodetectors and possibly multiple light sources emitting light in different bands, for measuring content of more than one target chemical at the same time. For example, combining light sources that measure water and fat contents, such a system could independently determine content of each independently.

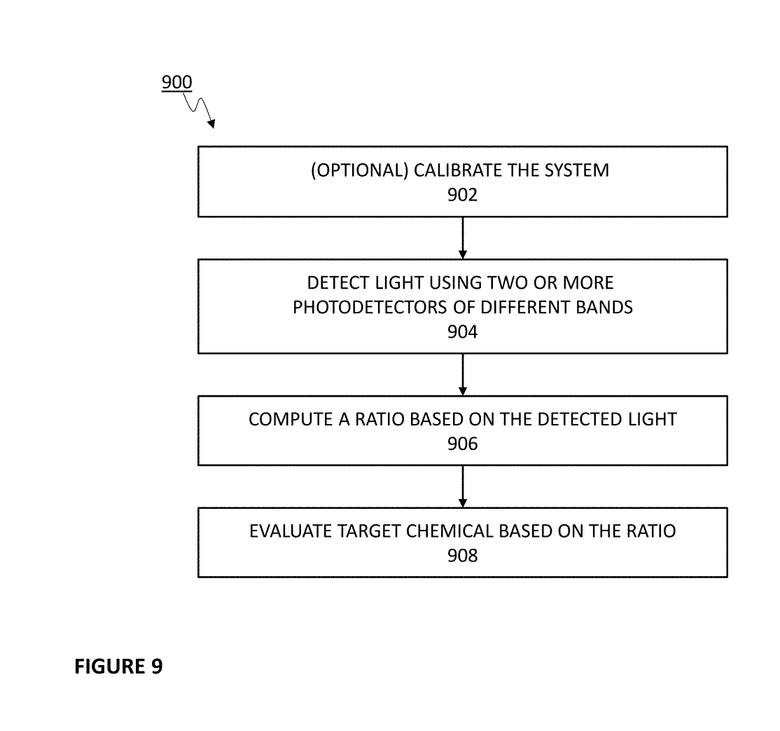

FIG. 9 illustrates a flow diagram of an optical detection method 900, according to some embodiments of the disclosure. Although described with reference to the system illustrated in FIG. 3, any systems configured to perform steps of method 900, in any order, are within the scope of the present disclosure.

At the beginning of the method, the system 300 may be calibrated (optional step 902). This may take place once, e.g. when the system 300 is being built or before the system 300 is put into operation, or may take place multiple times. During calibration, a plurality of ratios for a plurality of samples having a known presence and/or a known amount of one or more of predefined target chemicals or artificial optical filters that mimic the target chemical(s) may be computed, as described above. Each ratio could be a ratio between at least one parameter indicative of at least the intensity, e.g. normalized intensity, of light that has interacted with the predefined target chemical as measured by one photodetector and another parameter indicative of at least the intensity, e.g. normalized intensity, of light as measured by another photodetector 302. Calibration could also include storing the plurality of computed ratios in association with identifications of the plurality of samples (i.e. identifying, for each computed ratio, the particular predefined target chemical for which the ratio was computed, as well as the known presence and/or the known amount of the predefined target chemicals in/on a sample for which the ratio was computed).

In operation, two or more photodetectors 302 would be used to detect light that has interacted with the target chemical (step 904). Results of photodetector measurements would be provided to the processing logic 304, which would then compute one or more ratios based on the intensities detected by the different photodetectors (step 906). Since the ratios are selected to be representative of the presence and/or the amount of the target chemical, the processing logic 304 could then evaluate the presence and/or the amount of the target chemical based on the computed ratio (step 908).

Embodiments described herein allow providing a relatively low cost alternative to a full spectrometer for monitoring target chemicals. Systems proposed herein are simpler than a spectrometer because they are compact, and are directly able to measure the target chemical and use fewer, at least two, wavelengths for the analysis. Employing two photodetectors with appropriate calibration procedure and computation of ratios or ratio of ratios provides independence from gain as well as allows smoothly changing background reflectance as described herein. Resulting modules may be made compact and low power. By rapidly modulating the light source(s), effects of ambient light may be eliminated. Furthermore, systems proposed herein are easy to calibrate against a standard and adapted to the target chemical and the test object (i.e. the object/sample in/on which the target chemical is provided), and more than one chemical measurement may be carried out by using more than one light source and/or multiple photodetectors.

### Selected Examples

Example 1 provides an apparatus for optical detection of a presence and/or an amount of a target chemical. The apparatus includes a first photodetector configured to detect light of a first wavelength that has interacted with the target chemical, and a second photodetector configured to detect light of a second wavelength that has interacted with the target chemical, the second wavelength being different from the first wavelength. The apparatus further includes a processing logic configured to compute a ratio (R) between a first parameter indicative of at least an intensity of the light detected by the first photodetector (Int1) (possibly indicative of a combination of intensities of the light detected by each of the first and second photodetectors) and a second parameter indicative of at least an intensity of the light detected by the second photodetector (Int1), and determine the presence and/or the amount of the target chemical based on the computed ratio.

Example 2 provides the apparatus according to Example 1, where the first photodetector is configured to detect light of a first band of wavelengths that has interacted with the target chemical, the first band of wavelengths including the first wavelength, and the second photodetector is configured to detect light of a second band of wavelengths that has interacted with the target chemical, the second band of wavelengths including the second wavelength.

Example 3 provides the apparatus according to Example 2, where the first band of wavelengths and the second band of wavelengths at least partially overlap.

Example 4 provides the apparatus according to Example 3, where the first band of wavelengths is included within the second band of wavelengths and the ratio is computed as

$$R = \frac{α Int 1 - β ( Int 2 - Int 1 )}{Int 2} ,$$

where α and β are predefined parameters.

Example 5 provides the apparatus according to Example 2, where the first band of wavelengths and the second band of wavelengths do not overlap and the ratio is computed as

$$R = \frac{Int 2 - Int 1}{Int 2 + Int 1} , R = \frac{Int 2}{Int 1} , R = \frac{Int 2 + Int 1}{Int 2 - Int 1} , or R = \frac{Int 1}{Int 2} .$$

Example 6 provides the apparatus according to any one of the preceding Examples, where the first photodetector is provided at a distance less than 5 millimeters from the second photodetector.

Example 7 provides the apparatus according to any one of Examples 1-5, where each of the first photodetector and the second photodetector includes a plurality of photodetecting regions, and the photodetecting regions of the first photodetector are interleaved with the photodetecting regions of the second photodetector.

Example 8 provides the apparatus according to Examples 6 or 7, where the first photodetector and the second photodetector are provided on the same die.

Example 9 provides the apparatus according to any one of the preceding Examples, where the first photodetector is configured to detect the light of the first wavelength by detecting light incident on one or more photodetecting regions of the first photodetector that has passed through a first optical filter and the second photodetector is configured to detect the light of the second wavelength by detecting light incident on one or more photodetecting regions of the second photodetector that has passed through a second optical filter.

Example 10 provides the apparatus according to Example 9, where the first optical filter and/or the second optical filter is provided as a coating over a respective photodetector.

Example 11 provides the apparatus according to any one of the preceding Examples, where the light of the first wavelength is modulated and the light detected by the first photodetector is locked to the modulation of the light of the first wavelength, and/or the light of the second wavelength is modulated and the light detected by the second photodetector is locked to the modulation of the light of the second wavelength. In this manner, contamination from the ambient light at the first and/or second wavelengths may be reduced or eliminated.

Example 12 provides the apparatus according to any one of the preceding Examples, where the first photodetector is configured to detect light of the first wavelength substantially simultaneously with, or at least temporally overlapping, the second photodetector detecting light of the second wavelength. In this manner, potential motion induced artifacts, such as e.g. when measuring skin hydration on the hands and the hands not being held steady or when measuring moisture in industrial process control and samples being evaluated moving on a conveyer belt, may be reduced or eliminated.

Example 13 provides the apparatus according to any one of the preceding Examples, further including one or more light sources configured to generate light to interact with the target chemical, the light including at least the light of the first wavelength (or of the first band of wavelengths including the first wavelength) and the light of the second wavelength (or of the second band of wavelengths including the second wavelength).

Example 14 provides the apparatus according to Example 13, where the light generated by the one or more light sources includes broadband light (e.g. extended white light including wavelengths in the range of 1300-1600 nm or in the range of 1800-2000 nm for water measurements, in the range of 1600-1800 nm or in the range of 1100-1300 for fat or oil measurements, etc.).

Example 15 provides the apparatus according to Example 14, where a band of the light generated by the one or more light sources at least partially overlaps with a band of light that the first photodetector is configured to detect and with a band of light that the second photodetector is configured to detect.

Example 16 provides the apparatus according to any one of Examples 13-15, where the one or more light sources include a light emitting diode centered at about 1460 nm or/and a light emitting diode centered at about 1930 nm, and the target chemical includes, or is, water.

Example 17 provides the apparatus according to any one of Examples 13-15, where the one or more light sources include a light emitting diode centered at about 1200 nm, and the target chemical includes, or is, a fat.

Example 18 provides the apparatus according to any one of the preceding Examples, further including a third photodetector configured to detect light of a third wavelength that has interacted with the target chemical, the third wavelength being different from the first and the second wavelengths, where the processing logic determining the ratio includes the processing logic determining one or more ratios between the first parameter, the second parameter, and a third parameter indicative of at least an intensity of the light detected by the third photodetector (Int3), and the processing logic determining the presence and/or the amount of the target chemical based on the computed ratio includes the processing logic determining the presence and/or the amount of the target chemical based on the determined one or more ratios.

Example 19 provides a method for optical detection of a presence and/or an amount of a target chemical. The method includes computing a ratio (R) between a first parameter indicative of at least an intensity of light of a first wavelength that has interacted with the target chemical, detected by a first photodetector (Int1) (possibly indicative of a combination of intensities of the light detected by each of the first and second photodetectors) and a second parameter indicative of at least an intensity of the light of a second wavelength that has interacted with the target chemical, the second wavelength being different from the first wavelength, detected by a second photodetector (Int1); and determining the presence and/or the amount of the target chemical based on the computed ratio.

Example 20 provides the method according to Example 19, further including performing a calibration, prior to determining the presence and/or the amount of the target chemical, by computing a plurality of ratios for a plurality of samples having a known presence and/or a known amount of one or more of predefined target chemicals, each ratio including a ratio between the first parameter indicative of at least the intensity of light of the first wavelength that has interacted with the predefined target chemical and the second parameter indicative of at least the intensity of light of the second wavelength that has interacted with the predefined target chemical; and storing the plurality of computed ratios in association with identifications of the plurality of samples (i.e. identifying, for each computed ratio, the particular predefined target chemical for which the ratio was computed, as well as the known presence and/or the known amount of the predefined target chemicals in/on a sample for which the ratio was computed).

### VARIATIONS AND IMPLEMENTATIONS

It is noted that the illustrations in the FIGURES do not necessary represent true layout, orientation, sizing, and/or geometry of an actual apparatus/assembly for optical detection of a presence and/or an amount of a target chemical. It is envisioned by the disclosure that various suitable layouts can be designed and implemented for apparatus/assembly configured to detect a presence and/or an amount of a target chemical based on a computed ratio of parameters indicative of intensities of light measured by different photodetectors. Based on the descriptions provided above, a person of ordinary skill in the art can easily envision various further embodiments and configurations of determining present/content of a target chemical using photodetectors configured to detect light in different bands, all of which are within the scope of the present disclosure. To that end, FIGS. 2-9 can vary significantly to achieve equivalent or similar results, and thus should not be construed as the only possible implementation which leverages the use of ratios disclosed herein.

It is envisioned that the apparatus/assembly described herein and/or the associated processing modules can be provided in many areas including medical equipment, security monitoring, patient monitoring, healthcare equipment, medical equipment, automotive equipment, aerospace equipment, consumer electronics, and sports equipment, etc.

In some cases, the apparatus/assembly and/or the associated processing module can be used in professional medical equipment in a healthcare setting such as doctor's offices, emergency rooms, hospitals, etc. In some cases, the apparatus/assembly and/or the associated processing module can be used in less formal settings, such as schools, gyms, homes, offices, outdoors, under water, etc. The apparatus/assembly and/or the associated processing module can be provided in a consumer healthcare product.

In the discussions of the embodiments above, the capacitors, clocks, DFFs, dividers, inductors, resistors, amplifiers, switches, digital core, transistors, and/or other components can readily be replaced, substituted, or otherwise modified in order to accommodate particular circuitry needs. Moreover, it should be noted that the use of complementary electronic devices, hardware, software, etc. offer an equally viable option for implementing the teachings of the present disclosure. For instance, instead of processing the signals in the digital domain, it is possible to provide equivalent electronics that can process the signals in the analog domain.

In one example embodiment, any number of electrical circuits of the FIGURES may be implemented on a board of an associated electronic device. The board can be a general circuit board that can hold various components of the internal electronic system of the electronic device and, further, provide connectors for other peripherals. More specifically, the board can provide the electrical connections by which the other components of the system can communicate electrically. Any suitable processors (inclusive of digital signal processors, microprocessors, supporting chipsets, etc.), computer-readable non-transitory memory elements, etc. can be suitably coupled to the board based on particular configuration needs, processing demands, computer designs, etc. Other components such as external storage, additional sensors, controllers for audio/video display, and peripheral devices may be attached to the board as plug-in cards, via cables, or integrated into the board itself. In various embodiments, the functionalities described herein may be implemented in emulation form as software or firmware running within one or more configurable (e.g., programmable) elements arranged in a structure that supports these functions. The software or firmware providing the emulation may be provided on non-transitory computer-readable storage medium comprising instructions to allow a processor to carry out those functionalities. In some cases, application specific hardware can be provided with or in the processor to carry out those functionalities.

In another example embodiment, the electrical circuits of the FIGURES may be implemented as stand-alone modules (e.g., a device with associated components and circuitry configured to perform a specific application or function) or implemented as plug-in modules into application specific hardware of electronic devices. Note that particular embodiments of the present disclosure may be readily included in a system on chip (SOC) package, either in part, or in whole. An SOC represents an IC that integrates components of a computer or other electronic system into a single chip. It may contain digital, analog, mixed-signal, and often radio frequency functions: all of which may be provided on a single chip substrate. Other embodiments may include a multi-chip-module (MCM), with a plurality of separate ICs located within a single electronic package and configured to interact closely with each other through the electronic package. In various other embodiments, the target chemical detection functionalities described herein may be implemented in one or more silicon cores in Application Specific Integrated Circuits (ASICs), Field Programmable Gate Arrays (FPGAs), and other semiconductor chips.

Note that the activities discussed above with reference to the FIGURES are applicable to any integrated circuits that involve signal processing, particularly those that can execute specialized software programs, or algorithms, some of which may be associated with processing digitized real-time data to detect chemical content. Certain embodiments can relate to multi-DSP signal processing, floating point processing, signal/control processing, fixed-function processing, microcontroller applications, etc. In certain contexts, the features discussed herein can be applicable to medical systems, scientific instrumentation, wireless and wired communications, radar, industrial process control, audio and video equipment, current sensing, instrumentation (which can be highly precise), and other digital-processing-based systems. Moreover, certain embodiments discussed above can be provisioned in digital signal processing technologies for medical imaging, patient monitoring, medical instrumentation, and home healthcare. This could include pulmonary monitors, heart rate monitors, pacemakers, etc. Other applications can involve automotive technologies for safety systems (e.g., stability control systems, driver assistance systems, braking systems, infotainment and interior applications of any kind). In yet other example scenarios, the teachings of the present disclosure can be applicable in the industrial markets that include process control systems aiming to track vital signs to help drive productivity, energy efficiency, and reliability.

Note that with the numerous examples provided herein, interaction may be described in terms of two, three, four, or more parts. However, this has been done for purposes of clarity and example only. It should be appreciated that the system can be consolidated in any suitable manner. Along similar design alternatives, any of the illustrated components, modules, and elements of the FIGURES may be combined in various possible configurations, all of which are clearly within the broad scope of the present disclosure. In certain cases, it may be easier to describe one or more of the functionalities of a given set of flows by only referencing a limited number of electrical elements. It should be appreciated that the features of the FIGURES and its teachings are readily scalable and can accommodate a large number of components, as well as more complicated/sophisticated arrangements and configurations. Accordingly, the examples provided should not limit the scope or inhibit the broad teachings of the electrical circuits as potentially applied to a myriad of other architectures.

Note that in the present disclosure, references to various features (e.g., elements, structures, modules, components, steps, operations, parts, characteristics, etc.) included in “one embodiment”, “example embodiment”, “an embodiment”, “another embodiment”, “some embodiments”, “various embodiments”, “other embodiments”, “alternative embodiment”, and the like are intended to mean that any such features are included in one or more embodiments of the present disclosure, but may or may not necessarily be combined in the same embodiments.

It is also important to note that the functions related to measuring chemical content illustrate only some of the possible functions that may be executed by, or within, systems illustrated in the FIGURES. Some of these operations may be deleted or removed where appropriate, or these operations may be modified or changed considerably without departing from the scope of the present disclosure. In addition, the timing of these operations may be altered considerably. The preceding operational flows have been offered for purposes of example and discussion. Substantial flexibility is provided by embodiments described herein in that any suitable arrangements, chronologies, configurations, and timing mechanisms may be provided without departing from the teachings of the present disclosure. Note that all optional features of the apparatus described above may also be implemented with respect to the method or process described herein and specifics in the examples may be used anywhere in one or more embodiments.

The ‘means for’ in these instances (above) can include (but is not limited to) using any suitable component discussed herein, along with any suitable software, circuitry, hub, computer code, logic, algorithms, hardware, controller, interface, link, bus, communication pathway, etc. In a second example, the system includes memory that further comprises machine-readable instructions that when executed cause the system to perform any of the activities discussed above.

Although the claims are presented in single dependency format in the style used before the USPTO, it should be understood that any claim can depend on and be combined with any preceding claim of the same type unless that is clearly technically infeasible.

What is claimed is:

1. An apparatus for optical detection of a presence and/or an amount of a target chemical, the apparatus comprising:
a first photodetector configured to detect light of a first wavelength that has interacted with the target chemical;
a second photodetector configured to detect light of a second wavelength that has interacted with the target chemical; and
a processing logic configured to:
compute a ratio (R) between a first parameter indicative of at least an intensity of the light detected by the first photodetector (Int1) and a second parameter indicative of at least an intensity of the light detected by the second photodetector (Int2), and
determine the presence and/or the amount of the target chemical based on the computed ratio.

2. The apparatus according to claim 1, wherein:
the first photodetector is configured to detect light of a first band of wavelengths that has interacted with the target chemical, the first band of wavelengths including the first wavelength, and
the second photodetector is configured to detect light of a second band of wavelengths that has interacted with the target chemical, the second band of wavelengths including the second wavelength.

3. The apparatus according to claim 2, wherein the first band of wavelengths and the second band of wavelengths at least partially overlap.

4. The apparatus according to claim 3, wherein the first band of wavelengths is included within the second band of wavelengths and the ratio is computed as
$$R = \frac{α Int 1 - β ( Int 2 - Int 1 )}{Int 2} ,$$
where α and β are predefined parameters.

5. The apparatus according to claim 2, wherein the ratio is computed as
$$R = \frac{Int 2 - Int 1}{Int 2 + Int 1} , R = \frac{Int 2}{Int 1} , R = \frac{Int 2 + Int 1}{Int 2 - Int 1} , or R = \frac{Int 1}{Int 2} .$$

6. The apparatus according to claim 1, wherein the first photodetector is provided at a distance less than 5 millimeters from the second photodetector.

7. The apparatus according to claim 6, wherein the first photodetector and the second photodetector are provided on the same die.

8. The apparatus according to claim 1, wherein each of the first photodetector and the second photodetector comprises a plurality of photodetecting regions, and the photodetecting regions of the first photodetector are interleaved with the photodetecting regions of the second photodetector.

9. The apparatus according to claim 1, wherein the first photodetector is configured to detect the light of the first wavelength by detecting light incident on one or more photodetecting regions of the first photodetector that has passed through a first optical filter and the second photodetector is configured to detect the light of the second wavelength by detecting light incident on one or more photodetecting regions of the second photodetector that has passed through a second optical filter.

10. The apparatus according to claim 9, wherein the first optical filter and/or the second optical filter is provided as a coating over a respective photodetector.

11. The apparatus according to claim 1, wherein:
the light of the first wavelength is modulated and the light detected by the first photodetector is locked to the modulation of the light of the first wavelength, and/or
the light of the second wavelength is modulated and the light detected by the second photodetector is locked to the modulation of the light of the second wavelength.

12. The apparatus according to claim 1, wherein the first photodetector is configured to detect light of the first wavelength substantially simultaneously with the second photodetector detecting light of the second wavelength.

13. The apparatus according to claim 1, further comprising one or more light sources configured to generate light to interact with the target chemical.

14. The apparatus according to claim 13, wherein the light generated by the one or more light sources comprises broadband light.

15. The apparatus according to claim 14, wherein a band of the light generated by the one or more light sources at least partially overlaps with a band of light that the first photodetector is configured to detect and with a band of light that the second photodetector is configured to detect.

16. The apparatus according to claim 13, wherein the one or more light sources comprise a light emitting diode centered at about 1460 nm or/and a light emitting diode centered at about 1930 nm, and the target chemical comprises water.

17. The apparatus according to claim 13, wherein the one or more light sources comprise a light emitting diode centered at about 1200 nm, and the target chemical comprises a fat.

18. The apparatus according to claim 1, further comprising a third photodetector configured to detect light of a third wavelength that has interacted with the target chemical, wherein:
the processing logic determining the ratio comprises the processing logic determining one or more ratios between the first parameter, the second parameter, and a third parameter indicative of at least an intensity of the light detected by the third photodetector (Int3), and
the processing logic determining the presence and/or the amount of the target chemical based on the computed ratio comprises the processing logic determining the presence and/or the amount of the target chemical based on the determined one or more ratios.

19. A method for optical detection of a presence and/or an amount of a target chemical, the method comprising:
computing a ratio (R) between a first parameter indicative of at least an intensity of light of a first wavelength that has interacted with the target chemical, detected by a first photodetector (Int1) and a second parameter indicative of at least an intensity of the light of a second wavelength that has interacted with the target chemical, detected by a second photodetector (Int2); and
determining the presence and/or the amount of the target chemical based on the computed ratio.

20. The method according to claim 19, further comprising performing a calibration, prior to determining the presence and/or the amount of the target chemical, by:
computing a plurality of ratios for a plurality of samples having a known presence and/or a known amount of one or more of predefined target chemicals, each ratio comprising a ratio between the first parameter indicative of at least the intensity of light of the first wavelength that has interacted with the predefined target chemical and the second parameter indicative of at least the intensity of light of the second wavelength that has interacted with the predefined target chemical; and
storing the plurality of computed ratios in association with identifications of the plurality of samples.
